(19) United States
(12) Patent Application Publication
(10) Pub. No.: US 2024/0270631 A1
(43) Pub. Date: Aug. 15, 2024

(54) METHODS OF COATING SUBSTRATES

(71) Applicant: Glycome Biopharma Limited, Co Cork (IE)

(72) Inventors: Eoin Scanlan, Dublin (IE); Graham Smith, Co Cork (IE); Silvia Fogli, Co Cork (IE); Anna Testolin, Co Cork (IE); Paula Colavita, Dublin (IE)

(73) Assignee: Glycome Biopharma Limited, Co Cork (IE)

(21) Appl. No.: 18/567,081

(22) PCT Filed: Jun. 8, 2022
(86) PCT No.: PCT/EP2022/065599

§ 371 Date: Dec. 5, 2023

(30) Foreign Application Priority Data
Jun. 8, 2021 (GB) 2108188.0

(51) Int. Cl. C03C 17/00; C03C 17/32

(52) U.S. Cl. CPC C03C 17/003; C03C 17/32; C03C 2218/31; C03C 2218/32

(57) ABSTRACT

A method of coating a substrate surface, the method comprising: providing a substrate having a surface, optionally, treating at least a portion of the substrate surface with an oxidising agent, treating at least a portion of the substrate surface with a composition comprising a polysaccharide, oligosaccharide, polyol or mixture thereof, and incubating the treated substrate with the composition for a predetermined time. Also disclosed are substrates comprising such a coating, vessels comprising such coated substrates and medicals devices comprising such substrates.

### FIELD OF THE INVENTION

The present invention relates to methods of coating substrate surfaces, to coated substrates obtained thereby, and to methods of reducing protein aggregation on a substrate surface. The present invention also relates to vessels for fluids, medical devices and syringes comprising coated substrates. The substrate may be glass, in particular borosilicate glass.

### BACKGROUND OF INVENTION

Protein aggregation and denaturing in formulations of proteinaceous compositions contained in devices may occur and this causes problems in diagnostics, analysis and drug delivery. Control of protein aggregate formation and denaturing is problematic.

Non-specific protein adsorption is a complex event. The process is governed by the properties of the protein (e.g. structure, size, and distribution of charge and polarity), the properties of the material surface (e.g. charge, roughness, and state of surface energy) environmental conditions (e.g. pH, ionic strength and temperature) and the kinetics of the adsorption process.

Proteins may bind non-specifically at the surface of materials used during sample preparation, such as pipette tips, sample tubes, well plates and vials, which can result in loss of experimental accuracy. Regulatory guidelines require bioanalytical methods to be validated not only in terms of linearity, sensitivity, accuracy, precision, selectivity and stability, but also in terms of carryover. Carryover results from the nonspecific adsorption of analyte(s) to parts of the analytical system and thus introduces bias in both identification and quantification assays. Hence, linearity, sensitivity and repeatability of the analyses are negatively affected.

Disposable systems have gained increased acceptance for large scale storage during manufacturing and processing of recombinant proteins and monoclonal antibodies in liquid and frozen forms. Interactions between containers and pharmaceutical solutions is important: the physicochemical properties of container materials contribute toward maintaining the integrity and stability of drug substances. Adsorption of a protein on to a container surface may result in loss of protein potency within a solution arising from changes in concentration, protein denaturation and/or degradation. Protein aggregation and denaturing of pharmaceutical compositions (such as antibodies, proteins and other peptides, for example, erythropoietin, interferon-gamma, infliximab, etanercept, and adalimumab, all of which may be delivered in pre-filled in syringes) may also cause adverse immune response and has resulted in the withdrawal of some biopharmaceuticals from the market.

Surface modifications of the materials used to produce medical devices and vessels for delivery of compositions is one approach to attempt to mitigate the problem. Surface modifications of protein contacting materials used in manufacturing storage, e.g. ethylene vinyl acetate (EVA) copolymers and low-density polyethylene (LDPE), can potentially mitigate aggregate formation and protein adsorption thereby offering improved product quality and safety. Materials include glass or polymers (e.g. cyclic olefin polymers, COPs) that may be modified by applications of an inorganic coating on the surface that will be in contact with the composition.

WO-A-2020/092373 discloses a drug container having a thermoplastic wall, a PECVD (plasma-enhanced chemical vapor deposition) drug-contact coating, and a polypeptide composition contained in the lumen. The drug-contact coating is on or adjacent to the internal surface of the container, positioned to contact a fluid in the lumen, and consists essentially of SiOₓC_{y}H_{z}, a barrier to reduce corrosion.

US-A-2015/0126941 discloses a filled package comprising a vessel, a barrier coating a protective coating on the vessel, and a fluid composition contained in the vessel in order to increase the shelf life of the package. The barrier coating is of SiOₓ (x is 1.5 to 2.9). The protective coating comprises a layer of a saccharide to stop leaching.

There is a need to provide surfaces of materials that are less prone to adsorption of pharmaceutical compositions (including protein aggregation and denaturing) and do not suffer from the problems of the prior art.

It is an aim of the present invention to address this need.

### SUMMARY OF INVENTION

The present invention accordingly provides in a first aspect a method of coating a substrate surface, the method comprising: a) providing a substrate having a surface, b) optionally, treating at least a portion of the substrate surface with an oxidising agent, c) treating at least a portion of the substrate surface with a composition comprising a polysaccharide, oligosaccharide, polyol or mixture thereof, and d) incubating the treated substrate with the composition for a predetermined time.

Generally, any suitable substrate may be used in the method. For example, the substrate may comprise quartz, glass (for example silica lime glass or borosilicate glass). In other aspects, the substrate may comprise one or more polymers (e.g. EVA, polyolefin (for example polyethylene or polypropylene), a polyester (for example polyethylene terephthalate), a polycarbonate, or any combination or copolymer of any of these) may be used in the method, but preferably the substrate may comprise a cyclic olefin polymer or co-polymer. The polymer (for example the cyclic olefin polymer) may comprise, at least partially, recycled polymer.

Cyclic olefin polymers are useful as high temperature polymers with outstanding optical properties, good chemical and heat resistance, and excellent dimensional stability. The COP may be produced from cyclic olefin monomers such as norbornene, cyclopentadiene (CPD), and/or dicyclopentadiene (DCPD).

Glass substrates (e.g. borosilicate glass substrates) are useful because they are often used in procedures involving proteinaceous or oligonucleotide compositions that may be susceptible to protein adsorption and/or aggregation or oligonucleotide adsorption and/or aggregation.

Quartz substrates are useful because they may be used in microfluidic apparatus and other apparatus.

Surprisingly, use of a polysaccharide, oligosaccharide, polyol or mixture thereof as a coating significantly reduces protein adsorption and/or aggregation and may also reduce oligonucleotide adsorption and/or aggregation.

Where the substrate is a polymer, the composition may be applied above one or more other coating layers (except a layer of silica) already deposited on the polymer surface. Preferably, the polymer surface does not comprise a silica coating.

The composition may be applied directly to the substrate surface, usually needing no inorganic layers already deposited on the substrate surface. Thus, preferably, the method comprises treating the substrate surface directly.

Although, it is thought that a number of polysaccharides, oligosaccharides, polyols or mixtures thereof may be useful in the method, the polysaccharide etc. may comprise a hexose derived polysaccharide. The polysaccharides may be polyhydroxylated. Generally, the polysaccharides may provide a relatively hydrophilic surface (e.g. water contact angle below 80°, below 70°, below 60°, below 50°, or lower), preferably once applied to the substrate surface.

The preferred polysaccharide may be selected from dextran, cellulose, one or more polyols, dextrin, polygalacturonic acid, hyaluronic acid, or a combination of two or more of these polysaccharides.

Use of these polysaccharides, oligosaccharides, polyols or mixtures thereof is greatly advantageous because the inventors have determined they significantly reduce protein aggregation when applied to substrate, both glass and polymer, surfaces.

The oxidising agent preferably affects the surface of the substrate but preferably does not adversely affect the bulk of the substrate. The oxidising agent may comprise a peroxide, optionally may comprise hydrogen peroxide, optionally may comprise hydrogen peroxide in 30% w/w aqueous solution. Generally, peroxide and/or other oxidising agents may also be suitable, for example O₃, hydroxyl radicals, atomic oxygen, ozonated water, H₂O₂ with and without decomposition catalysts (e.g. Cu ions, Fe ions, manganese oxide), periodate, hypochlorite, and/or permanganate.

The predetermined time may be in the range 0.5 mins to 240 mins. Other optional ranges for the predetermined time may be 1 min to 120 min, 1 min to 60 min, 1 min to 30 min, 1 min to 20 min, or 1 min to 10 min.

Treating at least a portion of the substrate surface and/or incubation may be at a temperature in the range 10° C. to 90° C., optionally 10° C. to 70° C.

Treating at least a portion of the substrate surface and/or treatment during incubation may comprise mechanical, chemical or electromagnetic acceleration of the process e.g. by sonication, microwave or UV irradiation, and/or ion-catalysis.

The composition may be in aqueous solution. Thus, the composition may comprise water. One or more co-solvent(s) may also be present, if suitable.

In some embodiments, the composition may comprise an oxidising agent. The oxidising agent in the composition may comprise a peroxide, optionally may comprise hydrogen peroxide, optionally may comprise hydrogen peroxide in 30% w/w aqueous solution.

In some embodiments, the method may further comprise a step of treating the substrate with an aqueous basic solution of pH 7 to 14, preferably pH 9-14. This may be advantageous because it may improve protein rejection (or rejection of lipids, liposomes or oligonucleotides) from substrate surfaces. This step may be done after one or more of the steps a), b), c) or d) in the method.

The substrates obtained by the present method have significantly reduced protein aggregation.

The present invention accordingly provides in a second aspect a coated substrate obtainable by coating at least one surface of a substrate according to a method of the first aspect.

Optionally, the coated substrate does not comprise a silica coating.

The present invention accordingly provides in a third aspect a substrate having a coating on at least one surface, the coating comprising a polysaccharide directly contacting the surface of the substrate.

The substrate may comprise glass, quartz or polymer. The glass may comprise borosilicate glass. The polymer may comprise a cyclic olefin polymer.

The polysaccharide preferably comprises dextran, cellulose, polyols (e.g. hydrogenated hydrolysates, e.g. of starch), dextrin, polygalacturonic acid, hyaluronic acid, or a combination of two or more of these polysaccharides.

Coated substrates of the present invention have a further great advantage in that they enhance the thermal and intrinsic stability of compositions stored in contact with the coated surface (e.g. when compared with the uncoated surface or other materials).

Thus, the present invention provides in a fourth aspect, use of a vessel comprising a coated substrate according to the third aspect to store a pharmaceutical composition (optionally a peptide composition), thereby enhancing the intrinsic and/or thermal stability of the pharmaceutical composition.

The present invention accordingly provides in a fifth aspect a method of reducing lipid or liposome, protein or oligonucleotide aggregation or adsorption on a substrate surface, the method comprising: a) providing a substrate as discussed above and according to the second aspect, and b) contacting the surface with a lipid, liposome containing, proteinaceous or oligonucleotide composition.

As discussed above, this is advantageous because it provides for improved storage conditions e.g. allowing storage at higher temperature and/or for longer than previously.

The pharmaceutical composition may thus comprise a liposome containing composition, a nucleotide (e.g. an oligonucleotide) composition, or a pharmaceutical proteinaceous composition. The pharmaceutical proteinaceous composition may comprise a monoclonal antibody composition, or a peptide hormone.

In some embodiments, the pharmaceutical proteinaceous composition may comprise one or more of a vaccine (e.g. a vaccine comprising a peptide), erythropoietin, interferon (α-, β-, and/or γ-interferon), infliximab, etanercept, adalimumab, rituximab, infliximab, trastuzumab, insulin, glucagon, and/or a gonadotrophin.

The pharmaceutical composition may comprise an injectable composition. Examples of injectable compositions may include:
Abarelix-Depot (hormone);
AbobotulinumtoxinA Injection (Dysport);
Acetadote (Acetylcysteine Injection);
Actemra (Tocilizumab Injection);
Acthrel (Corticorelin Ovine Triflutate for Injection);
Actimmune (Interferon gamma-1b);
Adacel (vaccine);
Adalimumab (Humira);
Adenoscan (Adenosine Injection);
Aldurazyme (Laronidase);
Alglucerase Injection (Ceredase);
Alkeran Injection (Melphalan Hcl Injection);
ALTU-238 (human growth hormone);
Arzerra (Ofatumumab Injection);
Avastin (Bevacizumab);
Azactam Injection (Aztreonam Injection);
BayHepB (hepatitis b immune globulin human); antibody);
BayTet (Tetanus Immune Globulin (Human)); antibody);
Bexxar (Tositumomab) (antibody);
Blenoxane (Bleomycin Sulfate Injection; peptide antibiotic);
Botox Cosmetic (OnabotulinumtoxinA for Injection; protein);
BR3-FC (protein);
Briobacept (antibody);
BTT-1023 (antibody);
Byetta (Exenatide; peptide);
Campath (Altemtuzumab; antibody)
Canakinumab Injection (Ilaris; antibody)
Carticel; (chondrocytes cells)
Cathflo;(Alteplase; protein)
Cerezyme (Imiglucerase) (enzyme);
Certolizumab Pegol (Cimzia; antibody);
Choriogonadotropin Alfa, recombinant (r-hCG) for Injection (Ovidrel; peptide hormone);
Chorionic gonadotropin (hCG) for Injection (Pregnyl; Follutein; Profasi; Novarel; peptide hormone);
Clofarabine Injection (Clolar, Evoltra; purine nucleoside);
Colistimethate Injection (Coly-Mycin M); (polypeptide)
Corifollitropin alfa (Elonva; peptide hormone);
Copaxone (Glatiramer Acetate; mix of peptides);
Cubicin (Daptomycin Injection; cyclic lipopeptide);
Dacetuzumab (antibody);
Darbepoietin Alfa (protein);
DDAVP Injection (Desmopressin Acetate Injection peptide hormone);
Denosumab Injection (Prolia; antibody);
DMOAD (Disease-Modifying OsteoArthritis Drugs; class of compounds some of which are peptides);
Ecallantide Injection (Kalbitor; protein);
Engerix (vaccine);
Enbrel (etanercept; protein);
Epratuzumab (antibody);
Erbitux (Cetuximab; antibody);
Erythropoietin (peptide hormone);
Essential Amino Acid Injection (Nephramine) (mix of amino acids);
Fabrazyme (Agalsidase beta; enzyme);
Fluarix Quadrivalent (vaccine);
Fludara (Fludarabine Phosphate); (nucleotide analog derivative);
Follitropin Alfa Injection (Gonal-f RFF; Cinnal-f; Fertilex; Ovaleap; Bemfola; peptide hormone);
Follitropin Beta Injection (Follistim; Follistim AQ Cartridge; Puregon; peptide hormone);
Follitropin Delta Injection (Rekovelle; peptide hormone);
Forteo (Teriparatide (rDNA origin) Injection; peptide hormone);
Foscamet Sodium Injection (Foscavir);
Fuzeon (enfuvirtide; peptide);
GA101 (Obinutuzumab; antibody);
Ganirelix (Ganirelix Acetate Injection; peptide);
Gardasil (vaccine);
GC1008 (Fresolimumab; antibody);
Gemtuzumab Ozogamicin for Injection (Mylotarg); (antibody-drug conjugate)
Golimumab Injection (Simponi Injection; antibody);
GlucaGen (Glucagon; peptide hormone);
Havrix;(vaccine)
Herceptin (Trastuzumab; antibody);
hG-CSF (Human granulocyte colony-stimulating factor; protein);
Humalog (Insulin lispro; peptide hormone);
Human Growth Hormone;
Humegon (Human gonadotropin; peptide hormone);
Humulin (Insulin and analogues (modified form of insulin?), peptide hormone);
IncobotulinumtoxinA for Injection (Xeomin; protein)
Increlex (Mecasermin [rDNA origin] Injection); (human growth factor)
Infanrix; (vaccine)
Insulin (peptide hormone);
InsulinAspart [rDNA origin] Inj (NovoLog); (peptide hormone)
Insulin Glargine [rDNA origin] Injection (Lantus); (peptide hormone)
Insulin Glulisine [rDNA origin] Inj (Apidra); (peptide hormone)
Interferon alfa-2b, Recombinant for Injection (Intron A); (protein)
Interferon beta-1b, Recombinant for Injection (Betaferon; protein)
Iplex (Mecasermin Rinfabate [rDNA origin] Injec-tion); (human growth factor)
Iprivask (Desirudin for injection; protein);
Istodax (Romidepsin for Injection); (peptide)
Kepivance (Palifermin; keratinocyte growth factor);
Keratinocyte (epidermal cells);
KFG (keratinocyte growth factor);
Kineret (Anakinra; protein);
Kinlytic (Urokinase Injection; enzyme)
Kinrix; (vaccine)
Lente (L); (Insulin zinc; peptide hormone)
Leptin; (peptide hormone)
Levemir; (insulin analogue; peptide hormone)
Leukine (Sargramostim; protein)
Leuprolide Acetate injection (Lupron; peptide);
Levothyroxine (amino acid);
Lexiscan (Regadenoson Injection) (nucleoside);
Liraglutide injection (Victoza; peptide);
Lucentis (Ranibizumab Injection) (antibody);
Lumizyme; (Alglucosidase alfa; enzyme);
Lutropin alfa (LH) for injection (Luveris; peptide hormone);
Menactra (vaccine);
Menotropins for Injection (Menopur; Repronex; Pergonal; peptide hormones);
MetMab (Onartuzumab; antibody);
Miacalcin; (polypepide);
Mipomersen (Kynamro oligonucleotide);
Myozyme (Alglucosidase alfa) (enzyme);
NEO-GAA; (Avalglucosidase alfa enzyme);
Neupogen (Filgrastim; protein);
Novolin; (Novolin R: Insulin; Novolin N: Insulin isophane; peptide hormone);
NeoRecormon (Epoetin beta; protein);
NPH (N) (Humulin N; Novolin N; Isophane Insulin; peptide hormone);
Novolin 70/30 Innolet (70% NPH, Human Insulin Isophane Suspension and 30% Regular, Human
Insulin Injection); (peptide hormone);
Nplate (Romiplostim; protein);
Octreotide Acetate Injection (Sandostatin LAR; peptide);
Ocrelizumab (Ocrevus; antibody);
Orencia (Abatacept; antibody);
Osteoprotegrin (protein);
Oxytocin Injection (Pitocin; peptide hormone);
Panitumumab Injection for Intravenous Use (Vectibix; antibody);
Parathyroid Hormone; (peptide hormone);
Pediarix (vaccine);
Peginterferon (Peginterferon alfa-2a: Pegasys; Peginterferon alfa-2b: PEGintron; Sylatron);
Pegfilgrastim (Neulasta; Ristempa; protein);
Pegfilgrastim-cbqv (Udenyca; protein);
Pertuzumab (2C4; Omnitarg; Perjeta; antibody);
Pramlintide Acetate Injection (Symlin; Symlin pen (device for administration); peptide hormone);
R-Gene 10 (Arginine Hydrochloride Injection) (amino acid);
Raptiva (Efalizumab; antibody);
Recombivarix HB (vaccine);
Remicade (Infliximab; antibody);
Retrovir IV (Zidovudine Injection) (nucleoside);
rhApo2L/TRAIL (Dulanermin; protein);
Rituximab (MabThera; Rituxan; Truxima; antibody);
Roferon-A (Interferon alfa-2a; protein);
Somatropin for injection (Accretropin; Genotropin; Humatrope; Saizen; Norditropin; Valtropin);
Somatropin (rDNA origin) for Injection (Nutropin; Nutropin Depot; Nutropin AQ; Serostim LQ;
Onmitrope; Tev-Tropin);
Stelara Injection (Ustekinumab; antibody);
Stemgen (Ancestim; protein);
Telavancin for Injection (Vibativ; lipoglycopeptide);
Tenecteplase (Metalyse; TNKase; protein);
Thymoglobulin (Anti-Thymocyte Globulin (Rabbit); antibody);
Thyrogen (Thyrotropin Alfa for Injection; peptide hormone);
Trastuzumab-Dml (antibody-drug conjugate);
Travasol (Amino Acids (Injection));
Trelstar (Triptorelin Pamoate for Injectable Suspension; peptide);
Twinrix (vaccine);
Typhoid Vi-Polysaccharide Vaccine (Thyphim Vi; vaccine);
Urofollitropin for Injection (Bravelle; Fertinex; Fertinorm; Metrodin; peptide hormone);
Ultralente (U) (Extended Insulin Zinc; peptide hormone);
Vancomycin Hydrochloride (Vancomycin Hydrochloride Injection; glycopeptide);
VAQTA (vaccine);
Xolair (Omalizumab; antibody);
Zenapax (Daclizumab; antibody); and/or
Zevalin (Ibritumomab tiuxetan; antibody).

The present invention according provides in a sixth aspect a vessel for fluids comprising a substrate as discussed above and as discussed in the second aspect.

The vessel may be selected from a multi-well plate, a pipette, a bottle, a flask, a vial, an Eppendorf tube, and/or a culture plate.

The present invention is particularly useful for medical devices. Thus, the present invention accordingly provides in a seventh aspect a medical device comprising a substrate as discussed above and in the second aspect.

The medical device may be a tube, e.g. a dispensing tube, a vial, a channel and/or a syringe, for example a disposable syringe.

In one aspect, there may be provided a syringe, wherein the barrel of the syringe comprises a substrate (coated as discussed herein), and wherein the at least one surface of the substrate is the inner surface of the barrel.

This is advantageous because such a coating surprisingly reduces the glide force when the plunger of the syringe is depressed.

In this specification, and unless the context suggests otherwise, cyclic olefin polymers (COP) as referred to herein include cyclic olefin copolymers (COC). Proteinaceous compositions as referred to herein include peptides, oligopeptides, and/or polypeptides in a composition and may include additional components such as excipients (e.g. polysorbates, sugar compounds such as lactose, dextrin, glucose, sucrose, and/or sorbitol), salts, solvent (and/or co-solvents) and other non-proteinaceous active pharmaceutical components, and their formulations. Polysaccharide includes oligosaccharides, polyols or mixtures thereof.

### BRIEF DESCRIPTION OF FIGURES

Embodiments of the present invention will be described in more detail with reference to the accompanying Figures in which:

FIG. 1. (a) Quantitative determinations of adsorbed BSA-FITC at pristine TOPAS (TM) (TW) and ZEONOR (TM) (ZW) surfaces retained in the form of a hard layer (black bars) and a soft layer (grey bars). (b) Rinsing protocols developed to tailor assay sensitivity to hard layer (HL) and soft layer (SL).

FIG. 2. Summary of protein surface coverage determined at pristine and treated surfaces resulting from 2 mg mL⁻¹ BSA-FITC incubation experiments at COP surfaces.

FIG. 3. Comparison of emission data (ΔMFI) resulting from 2 mg mL⁻¹ BSA-FITC incubation experiments at COP surfaces obtained via microscopy. The pristine surface is used as reference 100% emission.

FIG. 4. Comparison of emission data (ΔMFI) resulting from 2 mg mL⁻¹ BSA-FITC incubation experiments at COP surfaces obtained via microscopy. The pristine surface is used as reference 100% emission.

FIG. 5. Summary of protein surface coverage determined at pristine and PGA-treated syringes resulting from 2 mg mL⁻¹ BSA-FITC incubation experiments.

FIG. 6. Summary of protein surface coverage determined at pristine and PGA-treated syringes resulting from 2 mg mL⁻¹ Insulin-FITC incubation experiments.

FIG. 7. (a) GATR-FTIR spectra of a Zeonor (TM)® coupon surface after rinsing with water (ZW) and after treatment in H₂O₂ at 50° C. for 30 min (ZP50). (b) UV-Vis absorbance spectra of a 1 mm Zeonor (TM)® coupon after rinsing with water only (ZW) and after treatment with H₂O₂ at 50° C. for 30 min (ZP50).

FIG. 8. (a) GATR-FTIR spectra of a Zeonor (TM)® coupon surface after rinsing with water (ZW) and after oxidising treatment via exposure to a UV/ozone lamp for 5 (ZU5) and 10 min (ZU10). (b) UV-Vis absorbance spectra of a 1 mm Zeonor (TM)® coupon after rinsing with water only (ZW), and after and after oxidising treatment via exposure to a UV/ozone lamp for 5 (ZU5) and 10 min (ZU10).

FIG. 9. Water contact angle measurement obtained at COP coupon surfaces after rinsing in water and undergoing a range of treatment conditions with and without PGA.

FIG. 10. Comparison between the surface composition of a coupon of TOPAS and syringe type S1, analysed by FTIR.

FIG. 11. Comparison between the surface composition of a coupon of Zeonor and syringe type S3, analysed by FTIR.

FIG. 12. Comparison between the surface composition of a coupon of Zeonex and a syringe type S3, analysed by FTIR.

FIG. 13. Comparison between the surface composition of a coupon of TOPAS and a syringe type S2, analysed by FTIR.

FIG. 14. Comparison between the surface composition of a coupon of Zeonor and syringe type S2, analysed by FTIR.

FIG. 15. Comparison between the surface composition of a coupon of Zeonex and syringe type S2, analysed by FTIR.

FIG. 16. Summary of protein surface coverage determined at pristine and treated surfaces resulting from 2 mg mL⁻¹ BSA-FITC incubation experiments at borosilicate glass surfaces.

### DETAILED DESCRIPTION

The studies herein use a fluorescently labelled globular protein, BSA-FITC to monitor the extent of protein surface adsorption at substrate surfaces.

The substrates investigated are glass (in particular borosilicate glass) and cyclo-olefin polymers (COP) materials.

BSA is typically used as an indicator of the ability of a surface to resist unspecific protein adsorption.

A second (fluorescently labelled) protein, Insulin-FITC, has been used to confirm the generality of the effect and its applicability to a therapeutic protein.

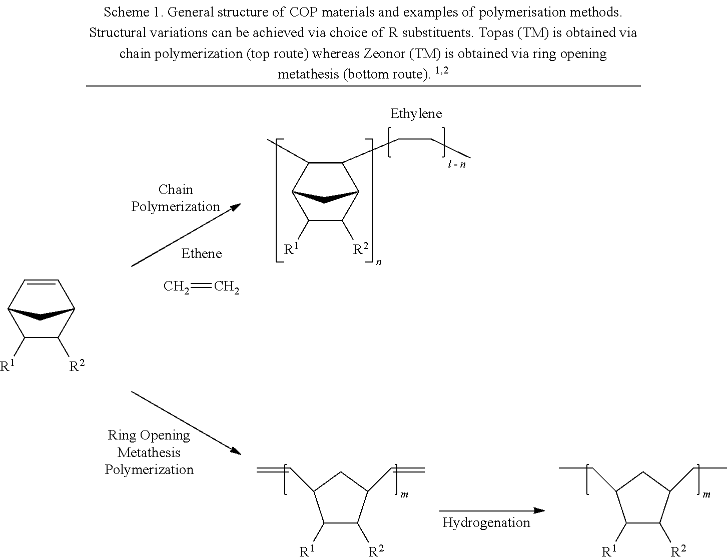

Three types of COP materials were investigated: TOPAS® (T) (Topas (TM) Advanced Polymer), ZEONOR® (Z) and ZEONEX® (Zeon Corporation) sourced from commercial suppliers in 1 mm thick coupon form. These materials are used by biodevice manufacturers for the biopharmaceutical industry. Scheme 1 shows a general structure of COP materials of different kinds; structural variations can be achieved via changes in the substituent groups which provide tunable properties. ¹ Shin J. Y. et al., Pure and Applied Chemistry, (2005) 77: 801-814.² Nunes et al. Microfluid Nanofluid (2010) 9:145-161.

To verify that the results of coupons were generalisable to biomedical devices, studies were conducted using selected syringe biodevices sold for pre-filled biotherapeutics sourced from three different manufacturers: Manufacturers #1-#3. All the syringes are of COP materials, while those manufactured by Manufacturer #1 are siliconized in their internal surface (barrel).

The adsorption of proteins to surfaces is a complex process; proteins typically undergo complete and/or partial denaturation when adsorbed at surfaces and the strength and nature of the interactions involved in protein adhesion varies.

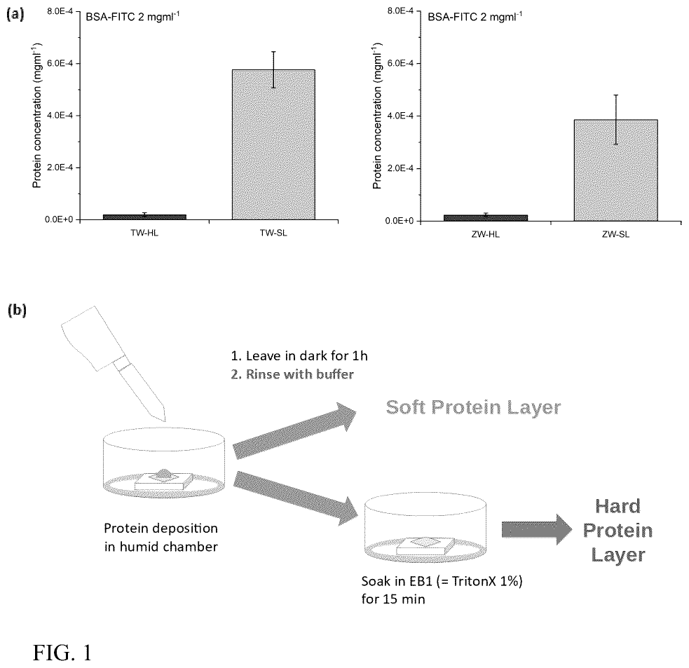

FIG. 1a shows quantitative determinations of the amount of BSA-FITC adsorbed at coupons of pristine Topas (TM) and Zeonor (TM).

Coupons (1.25 cm²) of these two COP materials were immersed in BSA-FITC solutions in phosphate buffer saline solution (PBS) at pH 7 at a concentration of 2 mg mL⁻¹ and incubated for 1 h in the dark to form BSA adlayers at the COP surface. Coupons were then rinsed in (method 1) PBS; or (method 2) in PBS and in elution buffer 1 (EB1=PBS+1% Triton X), as schematically depicted in FIG. 1b. Method 1 is expected to leave the largest amount of protein adsorbed, consisting of both soft and hard adsorbed layers of BSA. Method 2 is expected to remove most of the soft layer. After rinsing via methods 1&2, the adhered BSA-FITC was extracted into a 1 mL volume for quantitation via fluorescence methods. The extraction protocol consisted of incubation for 17 h in EB1 with addition of mercaptoethanol at 1% as a proteolytic agent, in order to fragment the protein and quantitatively release the FITC label into solution. The emission intensity from the extracted solution at 495 nm excitation was used to quantitate the protein via calibration with BSA-FITC standards.

The present study shows the effects of a surface modification using polysaccharides that shows significant promise in addressing protein adsorption.

Other work has shown that the protein rejection is observed also on the inner surface of syringes used for biotherapeutics, on COP materials. Protein rejection appears to be general, as it is observed with a general probe globular protein and with a therapeutic protein of smaller size.

### Examples: Polymer Substrates

Surface modification protocols. The surface modification protocols used 1.25 cm² coupons of TOPAS (TM) (T), ZEONOR (TM) (Z) and ZEONEX (ZX); these were subject to two different types of pre-treatment prior to modification with saccharides (id1 #in sample nomenclature):
1) Rinsing with millipore water (TW, ZW or ZXW)
2) Mild surface oxidation using hydrogen peroxide 30% at 50° C. (TP50, ZP50 or ZXP50). The pre-treated coupons were subsequently incubated in 1 mg mL⁻¹ solutions of different saccharides to carry out modifications of the surface. Scheme 2 shows the structures of polysaccharides tested in our experiments (id2 #in sample nomenclature): dextran (D), polygalacturonic acid (PGA), hyaluronic acid (H) or no saccharide (NS). The following incubation conditions were tested (id3 #in sample nomenclature):
1) Saccharide 1 mg mL⁻¹ in deionised water at room temperature for 2 h (W)
2) Saccharide 1 mg mL⁻¹ in deionised water at 50° C.; 4 consecutive incubations of 30 min (total 2 h) (W50X4).
3) Saccharide 1 mg mL⁻¹ in 30% H₂O₂ at 50° C.; 4 consecutive incubations of 30 min (total 2 h) (P50X4).

Following the incubation period, all samples were rinsed in deionised water and used for screening the amount of protein adsorption. To identify the treatment undergone by each surface tested, samples are referred by the combination of pre-treatment (id1 #), saccharide (id #2) and modification treatment (id3 #) used, as shown in Scheme 2.

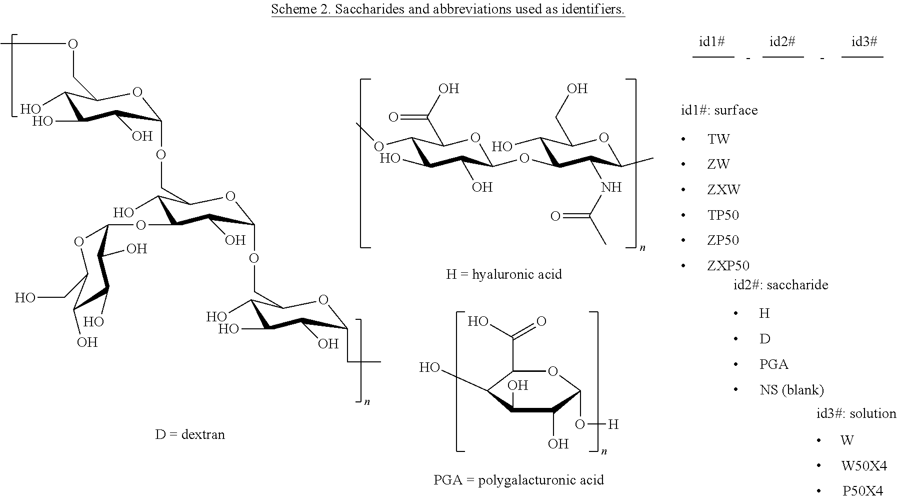

Protein adsorption testing protocol. Solutions of BSA-FITC were prepared in phosphate buffer saline solutions (PBS) at pH 7 at concentrations of 2 mg mL⁻¹. Coupons of the COP materials were immersed in BSA-FITC solutions and incubated for 1 h in the dark. The materials were then rinsed in (method 1) PBS and used for either quantitative or qualitative determinations as below:

a. Quantitative determinations via emission from solution. After rinsing the adhered BSA-FITC was extracted into a 1 mL volume for quantitation via fluorescence methods. The extraction protocol consisted of incubation for 17 h in EB1 with addition of mercaptoethanol at 1% as a proteolytic agent, in order to fragment the protein and quantitatively release the FITC label into solution. The emission intensity from the extracted solution at 470 nm excitation was used to quantitate the protein via calibration with BSA-FITC standards. The protein surface coverage was calculated by normalising the total extracted protein by the exposed COP area during incubation. Error bars in all graphs correspond to 95% C.I.

b. Qualitative comparisons via fluorescence microscopy. After rinsing the coupons were imaged using upright microscope with 470 nm excitation and a FITC exc/em filter cube to determine the integrated intensity at the COP surface via commercial software. Method 1 makes the method sensitive to both soft and hard adsorbed layers (FIG. 1). The mean fluorescence intensity (MFI) through the emission filter was measured from multiple images and corrected by the background emission (ΔMFI) of the corresponding pristine COP material. Error bars in all graphs correspond to 95% C.I.

### BSA-FITC Adsorption Results on COP Coupons

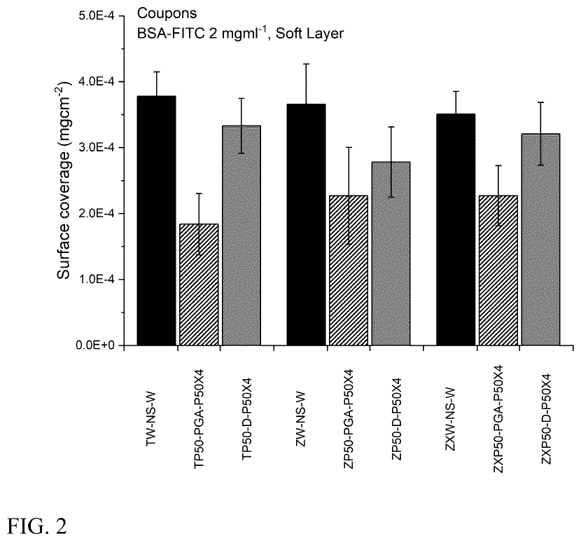

FIG. 2 shows results from quantitative determinations of BSA-FITC adsorption at Topas (TM), Zeonor (TM) and Zeonex surfaces. The ##-NS-W samples provide controls as it mimics the expected adsorption at e.g. a syringe barrel without any pre-treatment or modification. It is clear that modification with PGA polysaccharides yield the best reductions in the density of protein adsorbates. The best reduction is of 52% and observed for TP50-PGA-P50X4. Table A shows a summary of protein rejection results calculated as % adsorption relative to the pristine coupon surfaces.

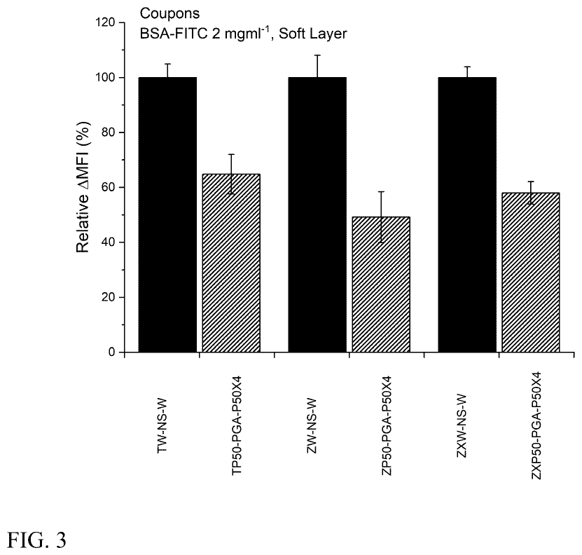

Protein adsorption changes were also confirmed via qualitative fluorescence microscopy methods as shown in FIG. 3. Emission from the coupon surface detected via microscopy shows that PGA-treatment results in lower emission from adsorbed BSA-FITC on all types of COP coupons tested.

TABLE A
Summary of results of protein rejection measurements
calculated from average values shown in FIG. 2.

| | TOPAS ™ | ZEONOR ™ | ZEONEX ™ |
|---|---|---|---|
| Polygalacturonic acid | 52% | 38% | 35% |
| Dextran | 13% | 24% | 7% |
| Hyaluronic acid | — | 8% | — |

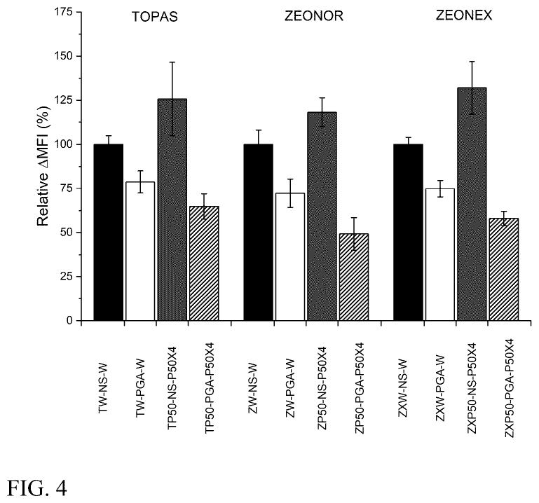

FIG. 4 shows the total emission from adsorbed BSA-FITC on the three polymer materials tested after the coupons were treated with PGA alone, with hydrogen peroxide alone or using the combination of PGA and peroxide treatment. It is evident that PGA alone does not result in as significant a reduction as when the surface is also treated with peroxide; whereas peroxide has a largely negative effect on protein rejection unless PGA is added to the treatment solutions.

### Protein Adsorption Results on COP Syringes

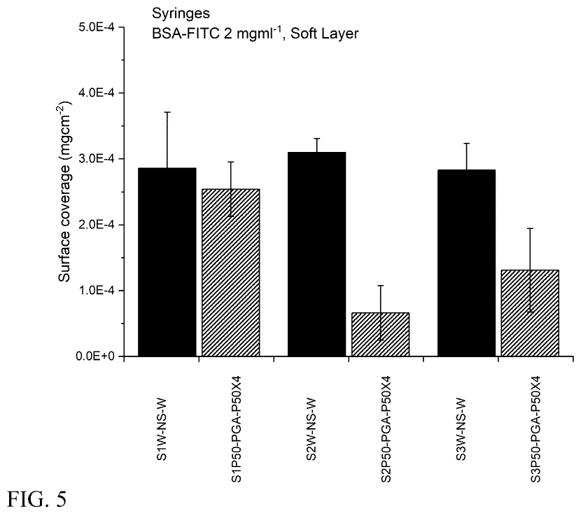

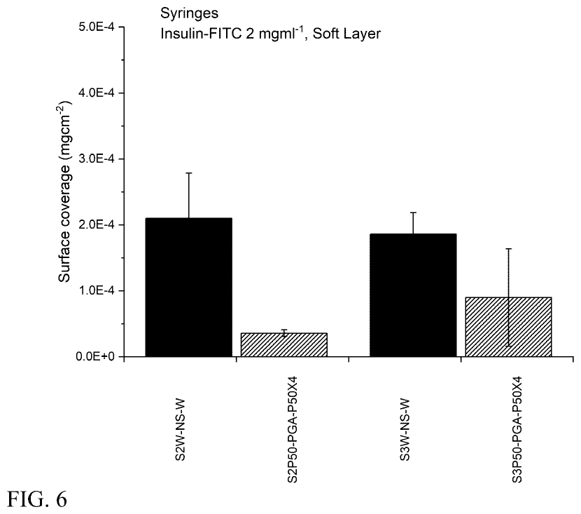

FIG. 5 shows results from quantitative determinations of BSA-FITC adsorption at Manufacturer #1, #2 and #3 COP syringes. The ##-NS-W syringes provide controls as they report the expected adsorption at clean syringe barrels without any pre-treatment or modification. It is clear that whereas pristine syringes display surface coverage of adsorbates that is comparable to that determined on coupon samples, the PGA modifications result in a significant reduction of BSA-FITC adsorption for #1 (79%) and #2 (54%) syringes. #1 syringes do not show significant reduction. However, this is consistent with these devices being siliconized over their inner surface and therefore indicate that COP surfaces are most affected by the polysaccharide treatment directly on their surface without a silica coating. Given the success of the modification protocol on #2 and #3 syringes we expanded the quantitative determinations to a different type of protein, Insulin-FITC, a protein that in its unlabelled form is used for therapeutic applications. FIG. 6 shows results from quantitative determinations with insulin-FITC; it is clear that also with this protein the PGA modification results in a decrease on #2 (83%) and #3 (52%) syringes.

### Effect of Surface Treatments on COP Materials

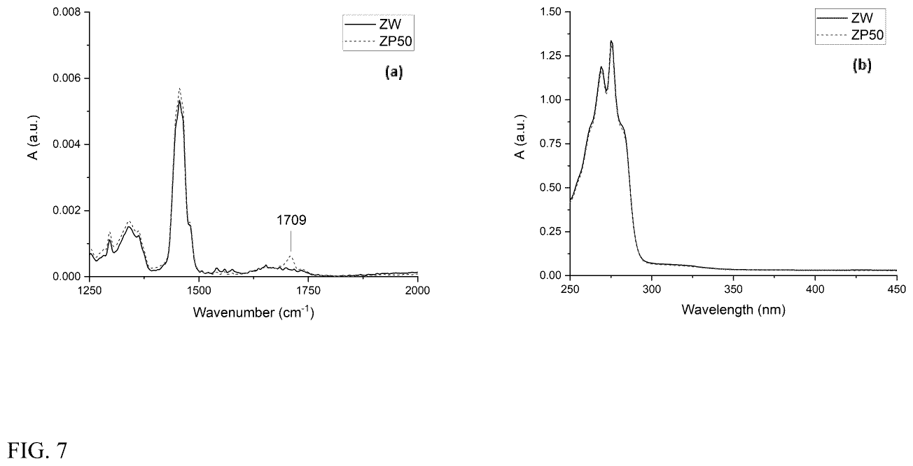

The effect of solution treatments and reactions conditions were investigated using Ge-attenuated total internal reflectance infrared spectroscopy (GATR-FTIR), water contact angle (WCA), and transmittance UV-Vis spectroscopy. FIG. 7a shows GATR-FTIR spectra of a COP coupon before and after exposure to H₂O₂ at 50° C.; the spectra show the appearance of a clear absorbance peak at 1709 cm¹ that is diagnostic of carbonyl functional groups. This indicates that exposure to peroxide at the reaction conditions results in oxidative activation of the COP. This oxidation is however mild and confined to the surface of the material as shown by control UV-Vis absorbance spectra in FIG. 7b, that indicates no change in the bulk optical properties.

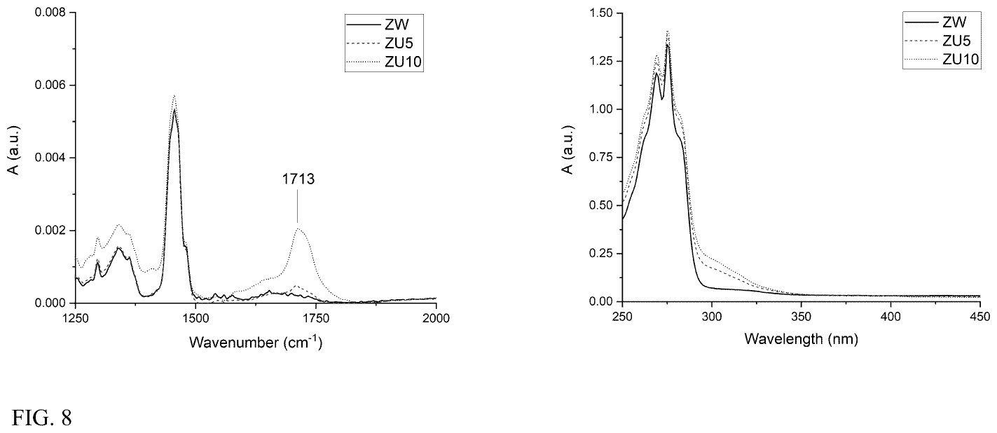

This is to be contrasted with other methods of surface oxidation such as exposure to UV/ozone lamp; this is shown in FIGS. 8a and 8b where the GATR and UV-Vis absorbances of the same type of COP coupon are shown after oxidation via UV/ozone lamp irradiation (10 min). Although the appearance of carbonyl peaks is apparent in the GATR-FTIR spectrum after oxidation, there is a significant increase in the optical absorbance in the UV-Vis absorbance spectrum, that is diagnostic of changes to the bulk structure of the COP polymer. The oxidation with H₂O₂ is therefore relatively mild and does not significantly alter the bulk material.

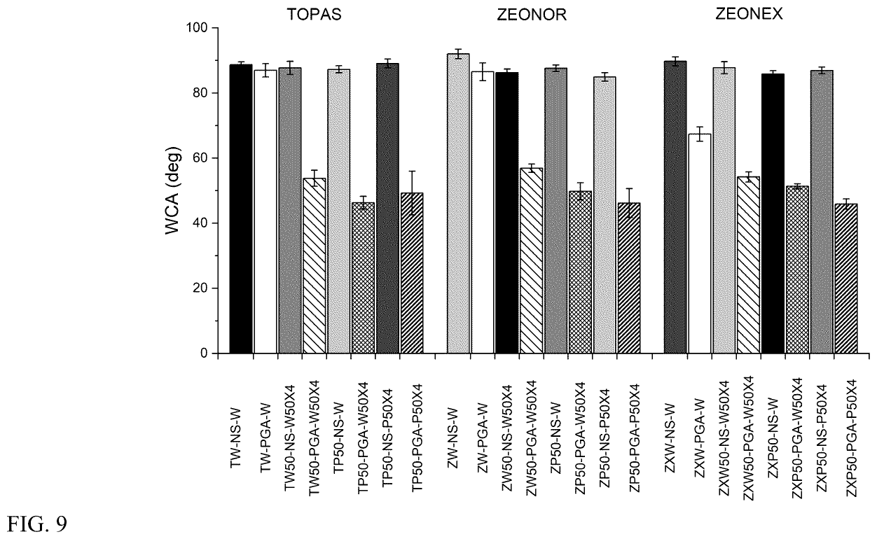

WCA measurements were used to monitor changes in hydrophilic character resulting from the surface treatments. FIG. 9 shows WCA values obtained at COP surfaces of the three polymers treated with and without PGA under different conditions. Results indicate that after H₂O₂ exposure alone only slight changes in hydrophilic character are observed; however, exposure to PGA results in significant increases in hydrophilic character.

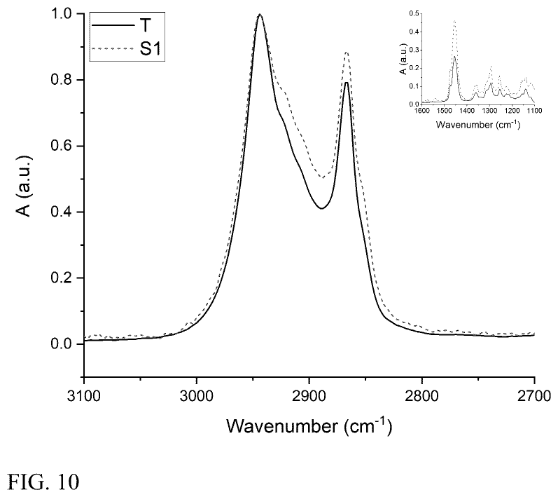

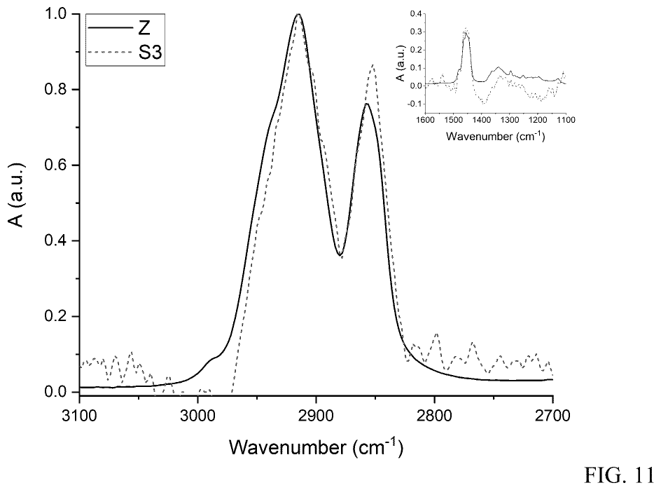

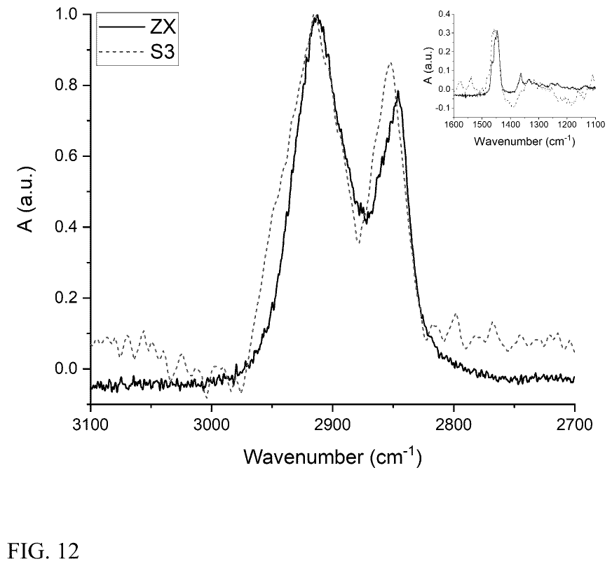

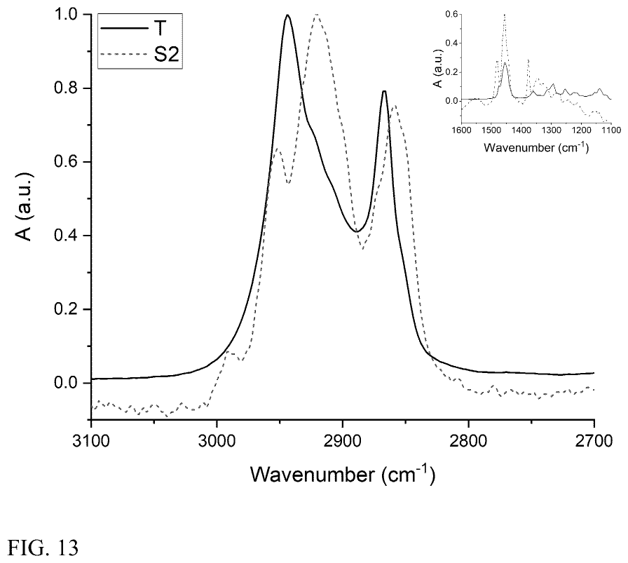

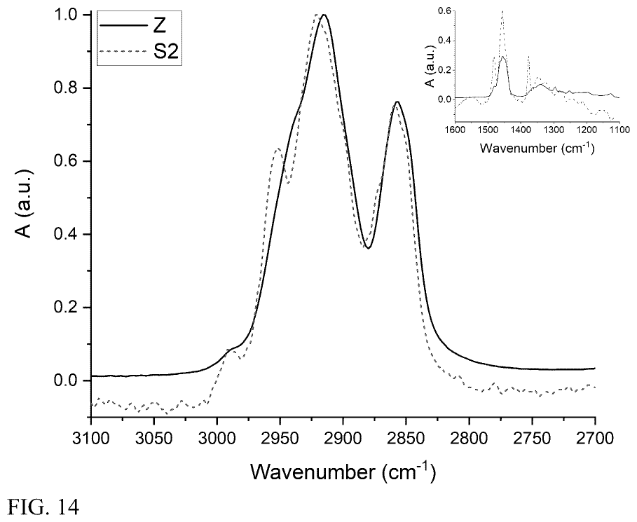

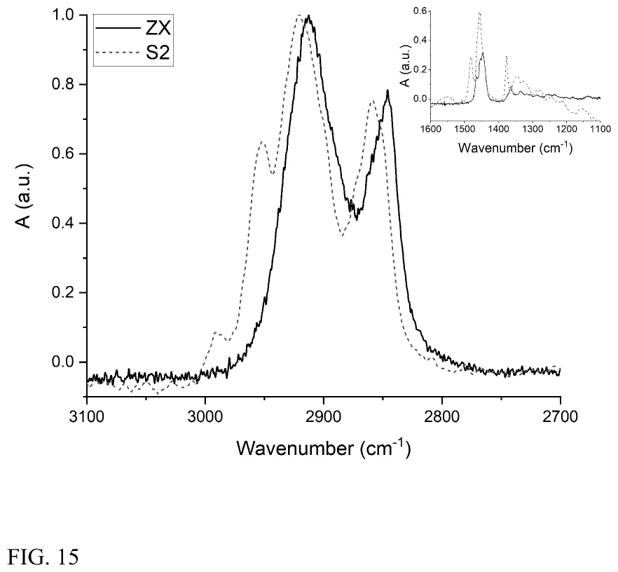

FIGS. 10 to 15 shows comparisons between the FT-IR spectra of COP materials (as coupons) and the syringe materials (types S1, S2, S3 from manufacturers #1, #2 and #3 respectively) discussed herein.

### CONCLUSION

For COP materials, a process of surface oxidation in combination with immobilization of a polysaccharide reduces still further protein adsorbates.

Protein rejection appears to be general, as it is observed with a general probe globular protein and with a therapeutic protein of smaller size.

### Example: Glass Substrate

Protein surface coverage was measured for untreated borosilicate glass and for modified borosilicate glass, obtained by two different procedures.

Untreated borosilicate glass was cleaned with acetone, isopropanol and deionized water before it was exposed to the protein.

Modified borosilicate glass, for both procedures, was subject to an oxidative treatment consisting of immersion in a Piranha solution (1 H₂O₂ 30% 3 H₂SO₄) for 45 minutes. The oxidative treatment may be replaced by or include a treatment step with an alkaline aqueous solution, generally with pH 7 to 14, optionally pH 9 to 14, optionally pH 10-14 at a temperature in the range 40° C. to 70° C.

This step was followed by a second oxidation, different for the two procedures:
1) P50: borosilicate glass was immersed in H₂O₂ 30% at 50° C. for 30 minutes.
2) U10: borosilicate glass was irradiated with UV-ozone lamp for 10 minutes on both sides.

Oxidative treatments were followed by functionalization with PGA, where borosilicate glass was immersed in a 1 mg/mL PGA solution in H₂O₂ 30% at 50° C. for 30 minutes. This step was repeated four times, changing PGA solution in peroxide after each cycle, for a total of 2 hours (PGA-P50X4).

Borosilicate glass surfaces were rinsed with deionized water prior their exposure to BSA protein.

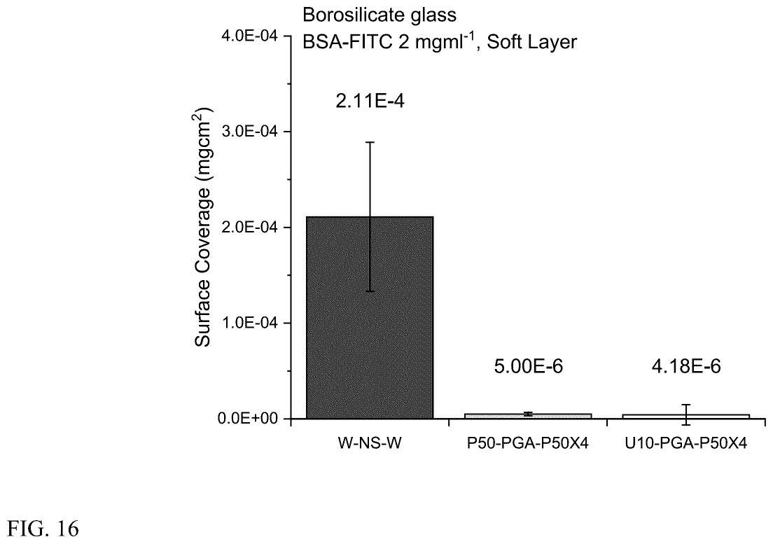

Adsorbed protein was quantitatively determined via emission from solution. After rinsing the adhered BSA-FITC was extracted for quantitation via fluorescence methods. The extraction protocol consisted of incubation for 17 h in EB1 with addition of mercaptoethanol at 1% as a proteolytic agent, in order to fragment the protein and quantitatively release the FITC label into solution. The emission intensity from the extracted solution at 470 nm excitation was used to quantitate the protein via calibration with BSA-FITC standards. The protein surface coverage was calculated by normalising the total extracted protein by the exposed area during incubation. Results of the investigation are shown in FIG. 16. Error bars in all graphs correspond to 95% C.I.

### Abbreviations

| | |
|---|---|
| PGA | Polygalacturonic acid |
| W | Substrate immersed in water at room temperature for 20′ × 3 times (1 h |
| | total) |
| P50 | Polymer substrate pre-treated with H₂O₂ 30% at 50° C. for 30′ |
| P70 | Glass substrate pre-treated with H₂O₂ 30% at 70° C. for 15 minutes |
| U10 | Substrate irradiated with UV-ozone lamp for 10′ |
| NS | No saccharide |
| S | Sugar (PGA, NS) |
| W-NS-W | W substrate, unmodified |
| W-S-W | W substrate immersed in sugar solution in water for 2 hours |
| P50-PGA- | P50 substrate immersed in sugar solution in H₂O₂ at 50° C. for 30' × 4 times |
| P50X4 | |
| P50-S-P50X4 | P50 immersed in sugar solution in H₂O₂ at 50° C. for 30 mins × 4 times |
| P70-S-P70X4 | P50 immersed in sugar solution in H₂O₂ at 70° C. for 15 mins × 4 times |
| U10-PGA- | U10 substrate immersed in sugar solution in H₂O₂ at 50° C. for 30′ × 4 |
| P50X4 | times |
| EB2 | Elution buffer 2: 1% Mercaptoethanol + 1% TritonX-100 in 2XSSPE |
| | buffer |
| BF | BSA-FITC solution in PBS buffer |

### Example COP, COC and Glass Substrate and Glide Force.

### Coating and Resistance Methods for Coupons and Syringes

### Solutions

1) PBS buffer: 4.58 g Na₂HPO₄+2.12 g NaH₂PO₄ in 1 L Millipore water
2) Sugars in Peroxide, 1 mg/mL (SP): 6 mg sugar in 6 mL H₂O₂ 30% for 4 times. The SP solution is prepared just before temperature treatment.
3) 2×SSPE buffer: 25 mL 20×SSPE buffer in 225 mL Millipore water
4) EB2: 0.5 mL TritonX-100+0.5 mL Mercaptoethanol in 49 mL 2×SSPE buffer
5) BF (2 mg/mL): 36 mg BSA-FITC in 18 mL PBS buffer

### Coating Procedure

1) Clean glass/polymer substrate according to the following procedures:
a) Millipore water (W): rinse substrate sequentially with Acetone, Isopropanol and finally Millipore water 2 times, changing water after each rinse. In the case of glass, the rinsing step might include a pre-rinse in acetone and alcohols (e.g. isopropanol) and a rinse in aqueous alkaline solution (e.g. NaOH) at pH 10-14 at 40-70° C.
b) Hydrogen Peroxide 30% T 50° C. (P50) or T 70° C. (P70): immerse samples in H₂O₂ and place for 15 minutes in water bath at 50° C. or 70° C.
2) Set aside W-NS-W substrates in water.
3) Place P50 or P70 pre-treated substrates in sugar solution and place at 50° C. for 30 minutes or 70° C. for 15 minutes (e.g. in a water bath). The solution may alternatively be sprayed onto the samples.
4) Repeat 4 times, changing the sugar solution after each cycle.
5) Rinse samples with fresh Millipore water for 3 times, changing water after each rinse.

### Resistance Tests:

pH
1) Place P50-S-P50X4 polymer or P70-S-P70X4 glass in the following conditions:
   a. pH 4: glass in 1 mL pH 4 solution for 48 hours at 4° C.
   b. pH 10: glass in 1 mL pH 10 solution for 48 hours at 4° C.

### Temperature

2) Place P50-S-P50X4 polymer or P70-S-P70X4 glass in the following conditions:
   a. −20° C.: wet glass at −20° C. for 1 week
   b. 4° C.: glass in 1 mL Millipore water for 1 week
   c. 20° C.: glass in 1 mL Millipore water for 1 hour
   d. 120° C.: wet glass in autoclave at 120° C. for 20 minutes

### Stress/Shear

3) Place P50-S-P50X4 polymer or P70-S-P70X4 glass in 0.5 mL Millipore water
4) Leave shaking at 500 rpm for 17 hours

### Incubation

5) Place P50-S-P50X4 polymer or P70-PGA-P70X4 glass in 1 mL BF
6) Leave at 4° C. for 1 week in the dark

### Storage

1) Place P50-S-P50X4 polymer or P70-PGA-P70X4 glass in 1 mL Millipore water
2) Leave at 4° C. for 1 week in the dark.

Protein adsorption quantitative determinations:

In this example, the method used is with fluorescence detection of eluted proteins.

Coupons of the material to be tested are cut to a known surface area. They are then immersed in a solution containing the formulation to be tested (e.g. buffer). A stock solution of protein-FITC conjugate relevant to the test (e.g. BSA-FITC) is pipetted to bring the solution to the desired protein concentration relevant to the test (e.g. 2 mg mL⁻¹). Coupons are incubated for 1 h in the dark at the temperature to be tested (e.g. 20° C.), to form protein adlayers. Coupons are then rinsed in phosphate buffer saline solution, pH 7, to remove excess/unbound conjugate. Coupons are subsequently incubated for 17 h in a known volume of elution buffer containing a detergent and a proteolytic agent to promote desorption and proteolysis of the surface-adsorbed protein-FITC. The fluorescence spectrum of the extracted solution is measured in a cuvette using a fluorimeter. The emission intensity at λ_{em,max} is used to determine protein concentration in the eluted volume via calibration with protein-FITC standards. If applicable, the eluted solution is diluted using PBS to bring the emission within the dynamic linear range and the dilution factor is used to determine total protein in the extracted volume. Finally, the total protein content extracted is normalised to the exposed surface area to calculate protein rejection values (Γₚᵣₒₜₑᵢₙ, %).

The results of test on coupons of COC, COP and Glass are set out in Tables 1 to 4 under shear/stress (500 rpm for 17 Hours), at varying pH at varying temperatures and over time. In the Tables, “PGA coating” refers to the substrate coated as set out above.

TABLE 1
Average calculated on N = 3 for COC and COP
Standard, N = 8 for COC and COP Shear/Stress,
N = 12 for Glass Standard and N = 6 for Glass Shear/Stress

Protein Rejection (%)

| | Standard | Shear/Stress |
|---|---|---|
| COC-PGA coating | 58 | 48 |
| COP-PGA coating | 44 | 44 |
| Glass-PGA coating | 95 | 97 |

TABLE 2
Average calculated on N = 3 for COC and COP at the
three pH, N = 12 for Glass Standard and N = 6
for Glass Shear/Stress and N = 4 for Glass pH 4 and pH 10

| | Protein Rejection (%) | | |
|---|---|---|---|
| | pH 4 | pH 7 | pH 10 |
| COC-PGA coating | 51 | 58 | 37 |
| COP-PGA coating | 41 | 44 | 36 |
| Glass-PGA coating | 94 | 95 | 100 |

TABLE 3
Average calculated on N = 15 for COC and COP at 20° C.,
N = 5 for COC and COP at 4° C., N = 3 for COC and
COP at −20° C., N = 12 for Glass at 20° C.,
N = 5 for Glass at 4° C., N = 6 for Glass at −20°
C. and N = 5 for COC and COP at 120° C.

Protein Rejection (%)

| | −20° C. | 4° C. | 20° C. | 120° C. |
|---|---|---|---|---|
| COC-PGA coating | 61 | 67 | 50 | — |
| COP-PGA coating | 54 | 76 | 28 | — |
| Glass-PGA coating | 89 | 91 | 95 | 89 |

TABLE 4
Average calculated on N = 15 for COC and COP
at t₀, N = 5 for COC and COP 1 week and 4 weeks,
N = 12 for Glass t₀ and N = 5 for Glass 1 week.

| | Protein Rejection (%) | | |
|---|---|---|---|
| | t₀ | 1 week | 4 weeks |
| COC-PGA coating | 50 | 34 | 38 |
| COP-PGA coating | 28 | 23 | 16 |
| Glass-PGA coating | 95 | 91 | — |

### Glide Force Measurement

Glide force was determined for syringes with a coated (PGA coating) or (as control) untreated inner surface of the syringe barrel with a plunger having a lubricated elastomeric tip. The syringes contained a test solution. The force to depress the plunger as a function of displacement was measured and the average force determined.

Tables 5 and 6 show results of glide force measurements (and standard deviation) for coated and uncoated syringes of COP1, COP2 and glass.

TABLE 5
Average calculated on N = 3 for Untreated COP1 and Glass syringes
and N = 5 for PGA coating COP1 and Glass syringes

Glide Force (N)

| | Untreated | PGA coating |
|---|---|---|
| COP1 | 5.99 ± 0.72 | 4.65 ± 0.39 |
| Glass | 6.58 ± 1.52 | 5.81 ± 1.05 |

TABLE 6
Average calculated on N = 3 for
Untreated and PGA coating on COP syringes

Glide Force (N)

| | Untreated | PGA coating |
|---|---|---|
| COP1 | 8.07 ± 0.56 | 6.94 ± 1.04 |
| COP2 | 8.37 ± 0.21 | 8.06 ± 0.46 |

### Roughness and Thickness of Coatings

AFM height profile determinations in air indicate a smooth topography with Rₐ=2.7±0.2 nm. Coating thickness via trench method d=4.5±0.7 nm

X-ray photoelectron spectroscopy determinations in UHV indicates chemical composition consistent with surface bound saccharide units. Average thickness via substrate attenuation method d=2.5 nm

### REFERENCES

1. (a) Gross, T., Ramm, M., Sonntag, H., Unger, W., Weijers, H. M., & Adem, E. H. Surf Interface Anal. 1992 18, 59; (b) Sawyer, Nesbitt & Secco J. Non-Cryst. Solids, 2012, 358, 290.
2. Jablonski & Zemek Surf Interface Anal. 2009, 41, 193
3. Briggs & Beamson Anal. Chem. 1992, 64, 1729
4. Clare, T. L., Clare, B. H., Nichols, B. M., Abbott, N. L. & Hamers, R. J. Langmuir 2005 21 (14), 6344.
5. Srinivasan & Nair, Clin.Mater. 1990, 6, 277.

The disclosures of the published documents referred to herein are incorporated by reference in their entirety.

1.-32. (canceled)

33. A method of coating a substrate, the method comprising;
a) providing a substrate having a surface,
b) optionally, treating at least a portion of the substrate surface with an oxidising agent,
c) treating at least a portion of the substrate surface with a composition comprising a polysaccharide, oligosaccharide, polyol or mixture thereof, and
d) incubating the treated substrate with the composition for a predetermined time.

34. The method of claim 33, wherein the substrate comprises quartz or glass.

35. The method of claim 34, wherein the substrate comprises borosilicate glass.

36. The method of claim 33, wherein the polysaccharide comprises a hexose derived polysaccharide or oligosaccharide.

37. The method of claim 33, wherein the polysaccharide comprises 20 percent or greater oxidised hexose at the C6 position.

38. The method of claim 33, wherein the polysaccharide is selected from dextrin, dextran polygalacturonic acid, hyaluronic acid, or a combination of two or more of these polysaccharide.

39. The method of claim 33, wherein the oxidising agent comprises a peroxide.

40. The method of claim 33, wherein the predetermined time is in the range 0.5 mins to 240 mins.

41. The method of claim 33, wherein the step of treating at least a portion of the substrate surface and/or step of incubating is at a temperature in the range 10° C. to 90° C.

42. The method of claim 33, wherein the composition comprises water.

43. The method of claim 33, wherein the composition comprises an oxidising agent.

44. The method of claim 43, wherein the oxidising agent in the composition comprises a peroxide, O₃, ozonated water, H₂O₂, periodate, hypochlorite, and/or permanganate.

45. The method of claim 33, further comprising a step of treating the substrate with an aqueous basic solution having a pH ranging from 9 to 14.

46. A substrate having a coating on at least one surface, the coating comprising a polysaccharide, oligosaccharide, polyol or mixture thereof directly contacting the surface of the substrate, wherein the substrate comprises quartz or glass.

47. The substrate of claim 46, wherein the substrate comprises borosilicate glass.

48. The substrate of claim 46, wherein the polysaccharide comprises dextrin, polygalacturonic acid, hyaluronic acid, or a combination of two or more of these polysaccharides.

49. A vessel comprising the substrate of claim 46, wherein the vessel is selected from a multi-well plate, a pipette, a bottle, a flask, a vial, an Eppendorf tube, and/or a culture plate.

50. A medical device comprising the substrate of claim 46, wherein the medical device is a dispensing tube, a vial, a device comprising a channel, or a syringe.

51. A method of reducing protein or oligonucleotide aggregation and/or adsorption on a substrate surface, the method comprising,
a) providing a substrate having a surface coating, the substrate comprising quartz or glass, and the surface coating comprising a polysaccharide, oligosaccharide, polyol or mixture thereof directly contacting the surface of the substrate; and
b) contacting the surface with a proteinaceous composition or a composition comprising an oligonucleotide.

52. The method of claim 51, wherein the proteinaceous composition comprises a pharmaceutical proteinaceous composition.
